(19) United States
(12) Patent Application Publication
(10) Pub. No.: US 2013/0261527 A1
(43) Pub. Date: Oct. 3, 2013

(54) PRESSURE GLOVE

(71) Applicants: Kit-Lun YICK, Hong Kong (HK); Zerance Sun-Pui NG, Hong Kong (HK); Joanne Yiu-Wan YIP, Hong Kong (HK)

(72) Inventors: Kit-Lun YICK, Hong Kong (HK); Zerance Sun-Pui NG, Hong Kong (HK); Joanne Yiu-Wan YIP, Hong Kong (HK)

(73) Assignee: THE HONG KONG POLYTECHNIC UNIVERSITY, Hong Kong (HK)

(21) Appl. No.: 13/433,268

(22) Filed: Mar. 28, 2012

(51) Int. Cl. A61F 13/10

(52) U.S. Cl. 602/53

(57) ABSTRACT

This invention discloses a therapeutic post-injury pressure glove and a method to produce the same. The pressure glove is configured to apply pressure onto a plurality of pressure-receiving regions of an injured hand such that each pressure-receiving region receives a pressure within an upper bound and a lower bound determined therefor. In one embodiment, the pressure glove comprises a plurality of custom pressure-applying fabric portions each of which is configured to direct a pressure onto one of the pressure-receiving regions. A suitable fabric specific for each fabric portion is selected so that adequate pressure is provided on a wounded area while maintaining a low pressure on an intact part of the hand. The pressure glove is fabricated with a size less than the hand's size by a reduction factor. The reduction factor and the set of selected fabrics are determined via obtaining a computed pressure distribution.

### FIELD OF THE INVENTION

The present invention relates to a therapeutic post-injury pressure glove personalized to a patient and configured to discourage hypertrophic scar development on the patient's hand that has an injury by applying pressure onto the hand.

### BACKGROUND

Hypertrophic scars are thick, raised, highly vascular and dark red tissues developed on the skin after having an injury such as burns and scalds. These scars may result in pain, cosmetic disfigurement, skin hypersensitivity and itchiness. Among various body parts, hands receive particular attention in hypertrophic scar management since finger and thumb joints are very sensitive and their freedom to move can be impaired in the presence of hypertrophic scars, causing a patient to become handicapped in serious cases.

Hypertrophic scars are typically developed during the wound healing process as a result of excessive deposition of collagen. It is desirable to discourage hypertrophic scar development. Herein hypertrophic scar development is referred to as formation of hypertrophic scars if not yet formed, and growth thereof if already present. A therapeutic method for discouraging hypertrophic scar development on an injured hand is by wearing a pressure glove. The pressure glove exerts sufficiently large pressure onto a wounded area in order to limit the blood flow into this area, thereby reducing the production of collagen.

To achieve a satisfactory therapeutic outcome, a patient is required to continually wear the pressure glove for 23 to 24 hours a day over a duration of 12-18 months or even until the scar matures in 2 or 3 years. Patient compliance is an important factor in the success of pressure-glove therapy. There have been reports on unsatisfactory performance in pressure-glove therapy due to failure in patient compliance. To encourage the patient to wear the pressure glove for a long time, it is required that the pressure glove enables the patient's hand to move freely and to feel comfortable. This requirement is as important as the requirement of providing adequate pressure onto a wounded area of the hand. One condition for satisfying the former requirement is that the pressure glove does not introduce excessively high pressure on an intact part of the hand.

Conventionally, a pressure glove for a patient is designed by the following approach. A suitable fabric type is first selected. According to geometric dimensions of the patient's injured hand, a glove pattern is developed, on which the pressure glove is fabricated with the selected fabric. The glove pattern has a size that is reduced by a certain percentage from the actual size of the hand. This percentage is referred to as a reduction factor. Practically, the reduction factor may be defined and measured as the percentage reduction of the length of the circumferential perimeter of the pressure glove with respect to the hand's circumferential perimeter. Typical values of the reduction factor range from 10% to 15% in accordance with the fabric extensibility. Pressure applied to the hand is induced by the stretching of the pressure glove when it is worn by the patient.

It is observed that in case the reduction factor is sufficiently high such that a wounded area receives adequate pressure, a comfortably low pressure on an intact part of the hand may not be guaranteed. Conversely, a low reduction factor may guarantee low induced pressure on the intact part, but may not result in adequate pressure on the wounded area for discouraging hypertrophic scar development. Designing a pressure glove by the aforementioned approach is therefore difficult to achieve the dual requirement that the pressure glove induces adequate pressure on the wounded area while maintaining a low pressure on the intact part of the hand.

It is desirable to have an improved pressure glove that can more easily satisfy the dual requirement than a pressure glove designed by the aforementioned approach.

### SUMMARY OF THE INVENTION

A first aspect of the present invention is a therapeutic post-injury pressure glove personalized to a patient and configured to apply pressure onto a plurality of pressure-receiving regions of the patient's hand having an injury such that each of the pres sure-receiving regions receives a pressure within an upper bound and a lower bound both determined for said each of the pressure-receiving regions. The pressure glove comprises a plurality of custom pressure-applying fabric portions each of which is configured to direct a pressure onto one of the pressure-receiving regions and is fabricated by a fabric. It results in a set of selected fabrics for fabricating the custom pressure-applying fabric portions. Furthermore, the pressure glove is fabricated with a size less than the hand's size by a reduction factor. The reduction factor and the set of selected fabrics are determined by obtaining a computed pressure distribution of the hand's surface under a simulated condition of wearing the pressure glove, and by checking the computed pressure distribution against the upper bounds and the lower bounds for all the pressure-receiving regions, so as to ensure that each of the pressure-receiving regions receives a pressure within the upper bound and the lower bound both determined for said each of the pressure-receiving regions. The computed pressure distribution is obtained by analyzing a bio-mechanical model of the hand and a mechanical model of the pressure glove. The bio-mechanical model of the hand incorporates three-dimensional (3D) geometric modeling details of the hand, and stiffness parameters of one or more of the hand's anatomical structures over the hand's surface. The pressure glove is personalized to the patient by at least obtaining the 3D geometric modeling details of the hand and the stiffness parameters of one or more of the hand's anatomical structures over the hand's surface by physical measurement on the hand. The mechanical model of the pressure glove incorporates geometric details of the custom pressure-applying fabric portions, and stiffness parameters of the fabrics selected for fabricating the custom pressure-applying fabric portions.

Optionally, the stiffness parameters of one or more of the hand's anatomical structures over the hand's surface comprise at least a tensile or compressive stiffness, a shear modulus and Poisson's ratio. It is also optional that the 3D geometric modeling details of the hand are acquired by a photogrammetric scanning system or other imaging system for performing physical measurement on the hand.

Preferably, at least two fabrics in the set of selected fabrics are substantially different in one or more aspects selected from composition, knitting structure, stress-strain and mechanical properties, comfort, air permeability, and deterioration of fabric elasticity, thereby allowing the pressure glove to be configured to achieve comfort and functional performance for the patient while discouraging hypertrophic scar development on the hand.

Preferably, at least one of the custom pressure-applying fabric portions is fabricated by a fabric that is a multi-layer fabric comprising at least a first fabric layer and a second fabric layer. In particular, the first fabric layer and the second fabric layer overlie each other and are joined together. Optionally, the first fabric layer and the second fabric layer are substantially different in one or more aspects selected from composition, knitting structure, stress-strain and mechanical properties, comfort, air permeability, and deterioration of fabric elasticity. In one embodiment, the first fabric layer and the second fabric layer are substantially similar in size. In another embodiment, the first fabric layer and the second fabric layer are substantially different in free size before being joined together. It allows a custom pressure-applying fabric portion fabricated by a multi-layer fabric having such first and second fabric layers to direct a different pressure onto a pressure-receiving region when compared to a custom pressure-applying fabric portion fabricated by a multi-layer fabric having a first fabric layer and a second fabric layer of equal free size. Preferably, the free sizes of the first fabric layer and of the second fabric layer are co-determined with the reduction factor and the set of selected fabrics according to the computed pressure distribution.

A second aspect of the present invention is a method for producing a therapeutic post-injury pressure glove configured to discourage hypertrophic scar development on a patient's hand that has an injury, wherein the pressure glove comprises a plurality of custom pressure-applying fabric portions that partition the pressure glove. In a first step of the method, a bio-mechanical model of the patient's hand is developed according to measured anatomical details of the hand including 3D geometric modeling details of the hand and stiffness parameters of one or more of the hand's anatomical structures over the hand's surface. In a second step, one or more pressure distributions of the hand's surface under a simulated condition of wearing a reference glove made of a reference material are predicted by analyzing the bio-mechanical model of the hand and a mechanical model of the reference glove. Each of the one or more predicted pressure distributions is obtained for a selected hand posture. The mechanical model of the reference glove incorporates geometric details of the reference glove and stiffness parameters of the reference material. By examining the one or more predicted pressure distributions, at least a plurality of high-pressure regions and a plurality of low-pressure regions on the hand's surface are identified in a third step of the method, so as to partition the hand's surface into a plurality of pressure-receiving regions. It results in a partitioning pattern of the hand. In a fourth step, an upper bound and a lower bound of pressure to be received from the pressure glove for each of the plurality of pressure-receiving regions are determined according to at least (1) wound-related details of the hand including locations, conditions and maturation of potential hypertrophic scars formed or potentially formed on the hand and (2) comfort to portions of the hand without such potential hypertrophic scars. Thereby, the pressure glove is configured to discourage hypertrophic scar development while providing comfort to the patient. In a fifth step, the pressure glove is partitioned into a plurality of custom pressure-applying fabric portions with a partitioning pattern substantially similar to the partitioning patterning of the hand. It follows that the pressure glove is designed to comprise the plurality of custom pressure-applying fabric portions each of which directs a pressure onto one of the pressure-receiving regions of the hand with said directed pressure within the upper bound and the lower bound of pressure determined for said one of the pressure-receiving regions. In a sixth step, a pressure glove having the custom pressure-applying fabric portions is produced, such that each of the custom pressure-applying fabric portions is configured to direct a pressure onto one of the pressure-receiving regions and within the upper bound and the lower bound of pressure determined therefor.

In producing the pressure glove, preferably, each of the pressure-applying fabric portions is fabricated by a fabric, and the pressure glove is fabricated with a size less than the hand's size by a reduction factor. A set of selected fabrics for fabricating the custom pressure-applying fabric portions is thereby obtained. Preferably, the sixth step of the method comprises: a first sub-step of determining the reduction factor and the set of selected fabrics; and a second sub-step of fabricating the pressure glove according to (1) geometric details of the custom pressure-applying fabric portions, and (2) the set of selected fabrics.

It is an option that at least one of the custom pressure-applying fabric portions is fabricated by a multi-layer fabric comprising at least a first fabric layer and a second fabric layer, both of which overlie each other and are joined together, with their free sizes substantially different before being joined together. Preferably, the sixth step of the method further comprises a third sub-step, performed before the second sub-step, of co-determining the free size of the first fabric layer and the free size of the second fabric layer with the reduction factor and the set of selected fabrics according to the computed pressure distribution.

### BRIEF DESCRIPTION OF THE DRAWINGS

FIG. 1 depicts an example of a pressure glove according to a first aspect of the present invention. This pressure glove comprises a plurality of custom pressure-applying fabric portions, each of which is configured to direct a pressure onto a pressure-receiving region of an injured hand.

FIG. 2 is a flowchart of a method for producing a therapeutic post-injury pressure glove according to a second aspect of the present invention.

### DETAILED DESCRIPTION

In the course of development of the present invention, it was observed that for a hypertrophic scar on an injured hand, the dimension, height, location and stiffness of the scar have an impact on the local pressure induced by a pressure glove on the tissue of the scar. For instance, a scar with a greater height can induce a greater local pressure since the local portion of the pressure glove is stretched to a greater extent, thereby providing greater compression force that acts on the scar. In another example, a scar located at the circumference of the hand receives a greater local pressure than another scar of the same height but located at the palm, since the compression force provided by the pressure glove is greater at a location having a greater curvature. A further example is that a less stiff scar generally receives a smaller local pressure.

The observation provides a basis that the hand's surface can be partitioned into a plurality of pressure-receiving regions, thereby resulting in a partitioning pattern of the hand. Each of the pressure-receiving regions receives a (local) pressure from the pressure glove. It follows that during the design phase of the pressure glove, a designer may assign a pressure to be generated by the pressure glove for a pressure-receiving region. More practically, the designer may specify an upper bound and a lower bound of pressure to be received by a pressure-receiving region as a design requirement.

Preferably, the upper bounds and the lower bounds of pressure for pressure-receiving regions overlapping with wounded areas of the hand can be determined by an occupational therapist according to locations, conditions and maturation of (potential) hypertrophic scars on these wounded areas and also according to the maximum pressure that can be tolerated by these scars without causing undesired physiological response such as pain. According to literature and experience of the inventors, a pressure in the range from 16.5 mmHg to 24.0 mmHg has been determined as effective for discouraging hypertrophic scar development. Accordingly, values of the lower bound and of the upper bound for the wounded areas may be set as 16.5 mmHg and 24.0 mmHg, respectively.

The upper bounds and the lower bounds of pressure for pressure-receiving regions on intact areas of the hand can be selected so as to enable the hand's fingers to move freely and the patient to feel comfortable. According to the present invention, maximum finger motility and patient comfort is achieved by setting the lower bound for the intact areas of the hand to 0 mmHg.

A first aspect of the present invention is a therapeutic post-injury pressure glove personalized to a patient and configured to apply pressure onto a plurality of pressure-receiving regions of the patient's hand that has an injury such that each of the pres sure-receiving regions receives a pressure within an upper bound and a lower bound both determined for said each of the pressure-receiving regions.

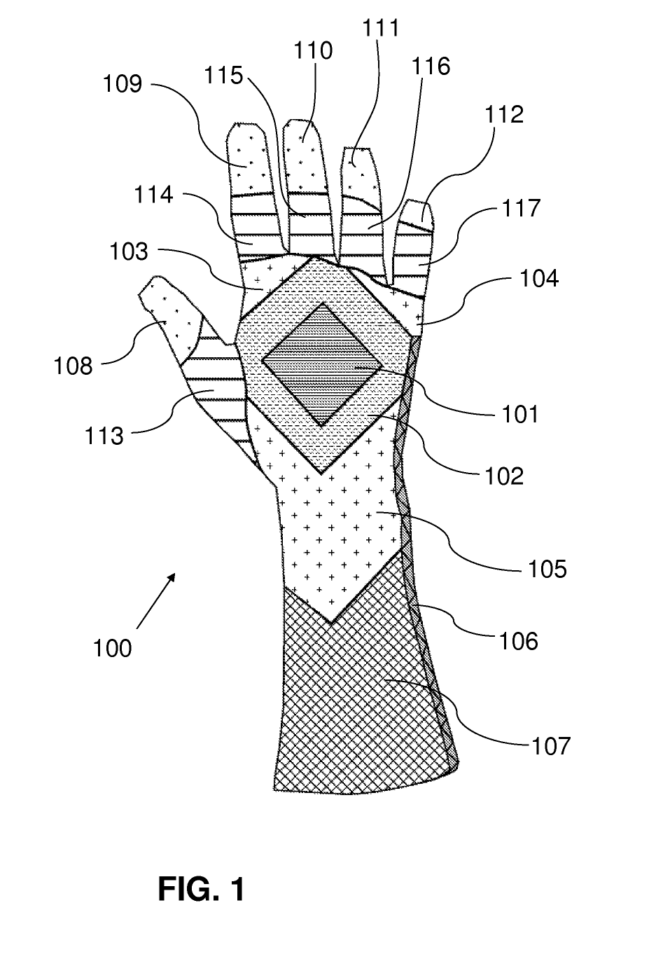

According to the first aspect of this invention, the pressure glove comprises a plurality of custom pressure-applying fabric portions each of which is configured to direct a pressure onto one of the pressure-receiving regions and is fabricated by a fabric. FIG. 1 depicts an example of such pressure glove. A pressure glove 100 is made up of custom pressure-applying fabric portions 101-117. In addition, the fabric portions 101-117 partition the pressure glove 100. By selecting a suitable fabric specific for each fabric portion among all fabric portions 101-117, the pressure glove 100 can be configured to provide adequate pressure on the wounded area while maintaining a low pressure on the intact part of the hand, thereby satisfying the dual requirement mentioned above. For instance, a stiff fabric and a soft fabric may be selected for fabricating, say, the fabric portion 101 and the fabric portion 109, respectively, where the fabric portion 101 is on a wounded area and the fabric portion 109 is on an intact part of the hand.

Furthermore, the pressure glove is fabricated with a size less than the size of the patient's hand by a reduction factor to thereby enable the pressure glove to generate pressure onto the hand by stretching of the pressure glove after it is applied to the hand. Typically, a greater reduction factor introduces more stretching than a lower reduction factor does, so that with a greater reduction factor, a higher pressure level is generally experienced on a location of the hand.

Since pressure levels induced by the pressure glove on the pressure-receiving regions depend on the reduction factor as well as geometric details and stiffness properties of the fabrics selected for fabricating the custom pressure-applying fabric portions, it follows that in the disclosed pressure glove, the reduction factor and the set of selected fabrics are determined so as to ensure that each of the pressure-receiving regions receives a pressure within the upper bound and the lower bound both determined for said each of the pressure-receiving regions. In particular, determining the reduction factor and the set of selected fabrics is done by obtaining a computed pressure distribution of the hand's surface under a simulated condition of wearing the pressure glove, and by checking the computed pressure distribution against the upper bounds and the lower bounds for all the pressure-receiving regions.

As mentioned above, the dimension, height, location and stiffness of a hypertrophic scar can affect the pressure levels induced by the pressure glove on the pressure-receiving regions. Therefore, geometric details and stiffness properties of the scar are involved in obtaining the computed pressure distribution. The computed pressure distribution is obtained by a mathematical method according to Leung, Wing Yan (2009), “Evaluation of compression garment design factor and prediction of garment pressure on wearer,” M. Phil. thesis, The Hong Kong Polytechnic University, 2009, the disclosure of which is incorporated by reference herein. According to Leung (2009), a bio-mechanical model of the hand and a mechanical model of the pressure glove are developed. The bio-mechanical model of the hand incorporates 3D geometric modeling details of the hand and stiffness parameters of one or more of the hand's anatomical structures over the hand's surface. The 3D geometric modeling details of the hand provide at least a numerical representation of the hand's surface in a 3D space. Examples of the hand's anatomical structures include skin, soft tissues, scar tissues, and nails, of the hand. The mechanical model of the pressure glove incorporates geometric details of the custom pressure-applying fabric portions and stiffness parameters of the fabrics selected for fabricating the custom pressure-applying fabric portions. The geometric details of the custom pressure-applying fabric portions provide at least a numerical description of shape and size of each of the custom pressure-applying fabric portions. Preferably this numerical description is specified for a two-dimensional plane. Specifying such numerical description for the two-dimensional plane is consistent with common industrial practice in that a design of a pressure glove is usually specified as a two-dimensional pattern. In the simulated condition of wearing the pressure glove, the shape and size of the pressure glove in a 3D space are determined by the 3D geometric modeling details of the hand.

The pressure glove is personalized to the patient by at least obtaining the 3D geometric modeling details of the hand and the stiffness parameters of one or more of the hand's anatomical structures over the hand's surface by physical measurement on the hand. The computed pressure distribution is obtained by analyzing a bio-mechanical model of the hand and the mechanical model of the pressure glove. Herein in the specification and in the appended claims, “analyzing” has a meaning of “analyzing by a mathematical technique” wherein the mathematical technique is an analytical technique, a numerical computation technique, a simulation technique, or a combination thereof.

As the pressure glove is fabricated with a size less than the hand's size by the reduction factor, it follows that the custom pressure-applying fabric portions have their sizes also affected by the reduction factor. Since the geometric details of the custom pressure-applying fabric portions include the sizes of these fabric portions, the mechanical model of the pressure glove incorporates a choice of the reduction factor. Hence, this choice of the reduction factor is involved in obtaining the computed pressure distribution, which is then used to check whether this choice of reduction factor, together with the set of selected fabrics, can satisfy a requirement that each of the pressure-receiving regions receives a pressure within the upper bound and the lower bound both determined for said each of the pressure-receiving regions. It follows that the reduction factor may be determined, for example, by an iterative process. A first choice of the reduction factor is selected and then tested if a resultant computed pressure distribution satisfies such requirement. If not, a second choice of the reduction factor is selected and tested again. This select-and-test procedure repeats until a choice that satisfies such requirement is obtained. Meanwhile, one or more fabrics may also be changed to finely adjust local pressure levels produced by such one or more fabrics on pressure-receiving regions in response to a change of the reduction factor.

It is preferable that the bio-mechanical model of the hand and the mechanical model of the pressure glove are represented in terms of finite element models, and that analyzing both models is done by a finite element analysis. Examples of building such finite element models and performing the finite element analysis are shown in Leung (2009). Optionally, the stiffness parameters of one or more of the hand's anatomical structures over the hand's surface may comprise at least a tensile or compressive stiffness, a shear modulus and Poisson's ratio. It is also optional that the stiffness parameters of the fabrics selected for fabricating the custom pressure-applying fabric portions may comprise at least a tensile or compressive stiffness, a shear modulus and Poisson's ratio. As a fabric may be anisotropic and/or nonlinear in stiffness properties, stiffness parameters of this fabric may vary along different directions that the fabric is stretched, and/or may change continuously for different degrees of stretching. Optionally, the stiffness parameters of the selected fabrics may include parameters to model anisotropy and/or nonlinearity of the selected fabrics. As detailed in Leung (2009), the stiffness parameters of one or more of the hand's anatomical structures over the hand's surface can be physically measured by a dial gauge with a miniature load cell, and the stiffness parameters of a fabric in the set of selected fabrics can be measured by a Kawabata Evaluation System for Fabric (KES-F). As an example, the 3D geometric modeling details of the hand can be acquired by a photogrammetric scanning system or other imaging system for performing physical measurement on the hand. The photogrammetric scanning system is detailed in Leung (2009).

In the selection of fabrics for fabricating the custom pressure-applying fabric portions, examples of candidate fabrics for selection include nylon/spandex/cotton blended fabrics of powernet, satinet and/or sleeknet structures. A further example of candidate fabric is a multiple-layer fabric comprising a plurality of fabric layers, where each of the fabric layers is made of a nylon/spandex/cotton blended fabric. The fabric layers may be joined together to form the multi-layer fabric by spacer yarns, forming a 3D warp knitted spacer fabric. The 3D spacer fabric can be used to provide a high pressure. The 3D structure of the 3D spacer fabric can also act as a buffer to prevent moisture build-up in the microclimate between the pressure glove and a wounded area. For an intact area of the hand, a soft, supple, highly breathable fabric with less compressibility may be selected so that a high sweat evaporation rate can be achieved, thus offering comfort to the patient. Other examples of candidate fabrics include those reported by Macintypre, L., Mitchell, C., Baird, M., and Weedall, P. J. (1999), “Elastic fabrics for the treatment of hypertrophic scars—comfort and colour,” Technical Textiles International, April 1999, pp. 19-22, the disclosure of which is incorporated by reference herein.

Preferably, at least two fabrics in the set of selected fabrics are substantially different in one or more aspects selected from composition, knitting structure, stress-strain and mechanical properties, comfort, air permeability, and deterioration of fabric elasticity, thereby allowing the pressure glove to be configured to achieve comfort and functional performance for the patient while discouraging hypertrophic scar development on the hand. For a description on the above-mentioned fabric properties, see Denton, M. J., and Daniels, P. N. (ed.), Textile Terms and Definitions, 11th ed., The Textile Institute, 2002, the disclosure of which is incorporated by reference herein. Typically, a piece of stretchable fabric with high stiffness, or a multi-layer fabric, may be used to fabricate a custom pressure-applying fabric portion that is required to provide a high pressure. A soft, supple fabric may be employed for a fabric portion intended to provide a low pressure even after repeated uses and soakings of the pressure glove, so as to allow the hand and fingers thereof to retain sensitivity and freedom of movement in daily activities.

As is mentioned above, in order to provide adequate pressure for discouraging hypertrophic scar development, an option is that at least one of the custom pressure-applying fabric portions is fabricated by a fabric that is a multi-layer fabric comprising a plurality of fabric layers, i.e. comprising at least a first fabric layer and a second fabric layer. The first fabric layer and the second fabric layer overlie each other and are joined together. Both fabric layers may be substantially similar or substantially different in one or more aspects selected from composition, knitting structure, stress-strain and mechanical properties, comfort, air permeability, and deterioration of fabric elasticity.

In one embodiment, the first fabric layer and the second fabric layer are substantially similar in size. Typically, a size of the first or the second fabric layer is chosen to be a size of a custom pressure-applying fabric portion fabricated by the multi-layer fabric.

In another embodiment, the first fabric layer and the second fabric layer are substantially different in free size before being joined together. Herein in the specification and in the appended claims, a free size of a fabric layer is referred to as a size of this fabric layer without being stretched or compressed by an external force. Typically, either the first or the second fabric layer, but not both, is chosen with a free size substantially similar to a size of a custom pressure-applying fabric portion fabricated by the multi-layer fabric. As mentioned above, the pressure glove is fabricated with a size less than the hand's size by the reduction factor. It follows that typically, each of the custom pressure-applying fabric portions is stretched to a larger size after the pressure glove is applied to the hand. Consider a situation that a fabric layer having a free size smaller than a size of a custom pressure-applying fabric layer fabricated by a multi-layer fabric having such fabric layer. The percentage increase in size of the fabric layer is greater than the percentage increase in size of another fabric layer having a free size equal to the size of the custom pressure-applying fabric portion, so that the former fabric layer generates greater compression force to the hand than the latter one. Similarly, a fabric layer having a free size greater than a size of a custom pressure-applying fabric layer fabricated by a multi-layer fabric with such fabric layer generates smaller compression force to the hand than another fabric layer having a free size equal to the size of this custom pressure-applying fabric portion. Therefore, a compression force directed to the hand can be realized as desired by changing the free size of either the first or the second fabric layer. Preferably, the free sizes of the first fabric layer and of the second fabric layer are co-determined with the reduction factor and the set of selected fabrics according to the computed pressure distribution.

The pressure glove can be fabricated based on the geometric details of the custom pressure-applying fabric portions and the set of selected fabrics, possibly at least one of the selected fabrics being a multi-layer fabric comprising a plurality of fabric layers. The selected fabrics can be joined by using one or more seaming and construction methods, such as zig-zag stitches with flat seams, thermoplastic adhesive tape, etc. More seaming and construction methods are described by Laing, R. M. and Webster, J. (1998), Stitches and Seams, The Textile Institute, 1998, the disclosure of which is incorporated by reference herein.

It is optional that after fabrication of the pressure glove, pressure levels provided by the custom pressure-applying fabric portions are checked and verified by measurement using pressure sensors.

A second aspect of the present invention is a method for producing a therapeutic post-injury pressure glove configured to discourage hypertrophic scar development on a patient's hand that has an injury, wherein the pressure glove comprises a plurality of custom pressure-applying fabric portions that partition the pressure glove. In particular, the method enables determination of a partitioning pattern of the pressure glove.

This partitioning pattern is an important element in designing the pressure glove. The pressure glove is required to be strategically partitioned into the plurality of custom pressure-applying fabric portions such that a high pressure can be directed to a wounded area on the injured hand while a low pressure is exerted to an intact part of the hand. Note that the partitioning pattern of the pressure glove is specific to the patient's injured hand. As is mentioned above, local pressure levels induced by the pressure glove are influenced by the dimension, height, location and stiffness of a hypertrophic scar on the hand. The method takes into consideration presence of the hypertrophic scar on the hand in determination of the partitioning pattern. Furthermore, the Applicant has observed that different hand postures can lead to different pressure distributions even produced by a same pressure glove, implying that examining a plurality of pressure distributions for different hand postures is preferable in determining the partitioning pattern.

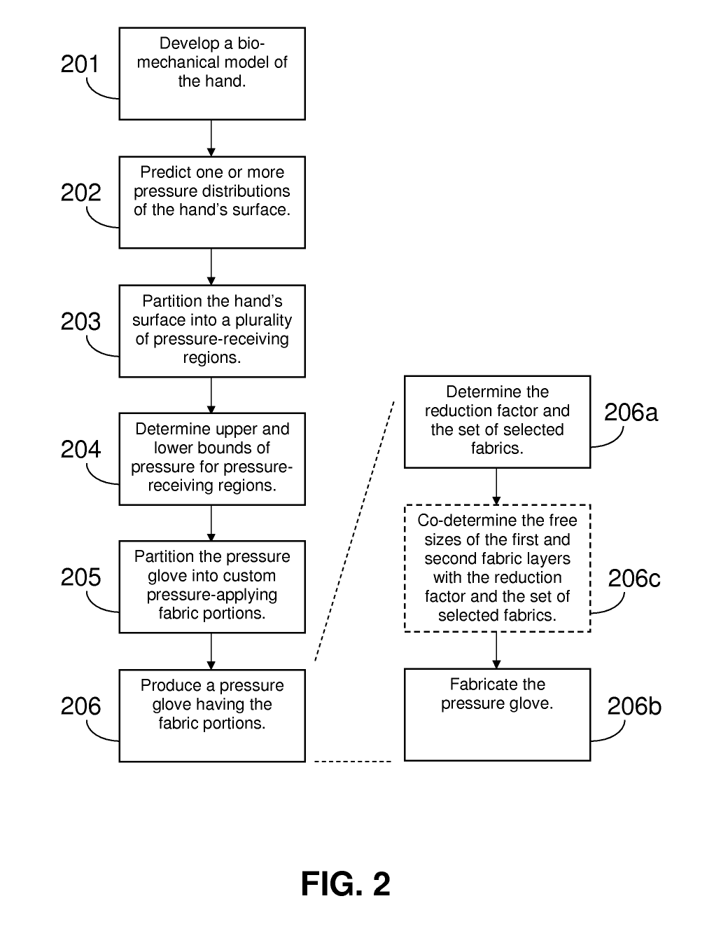

The method is elaborated as follows. FIG. 2 is a flowchart showing a sequence of steps taken in the method.

According to a first step 201 of the method, a bio-mechanical model of the hand is first developed. This bio-mechanical model is developed according to measured anatomical details of the hand including 3D geometric modeling details of the hand and stiffness parameters of one or more of the hand's anatomical structures over the hand's surface. As mentioned above, the 3D geometric modeling details of the hand provide at least a numerical representation of the hand's surface in a 3D space. The stiffness parameters of one or more of the hand's anatomical structures over the hand's surface may be obtained by measurement using a dial gauge with a miniature load cell as mentioned above and also as detailed in Leung (2009). Preferably, the stiffness parameters of one or more of the hand's anatomical structures over the hand's surface comprise at least a tensile or compressive stiffness, a shear modulus and Poisson's ratio. The 3D geometric modeling details of the hand may be acquired by a photogrammetric scanning system, as detailed in Leung (2009), or other imaging system.

A second step 202 of the method is to predict one or more pressure distributions of the hand's surface under a simulated condition of wearing a reference glove made of a reference material by analyzing the bio-mechanical model of the hand and a mechanical model of the reference glove, wherein each of the one or more predicted pressure distributions is obtained for a selected hand posture. The mechanical model of the reference glove incorporates geometric details of the reference glove and stiffness parameters of the reference material. A choice of the reference material can be any fabric, which may be, e.g., any one of the examples of candidate fabrics mentioned above. Preferably, the reference glove is selected according to a glove pattern selected for making the pressure glove. For example, if a fingerless glove pattern is intended for making the pressure glove in order to keep the hand cool and comfortable even in hot weather, the reference glove may adopt this fingerless glove pattern. The geometric details of the reference glove provide at least a numerical description of shape and size of the reference glove. Preferably this numerical description is specified for a two-dimensional plane.

In a third step 203 of the method, at least a plurality of high-pressure regions and a plurality of low-pressure regions on the hand's surface are identified by examining the one or more predicted pressure distributions. Based on results of this identification, the hand's surface is partitioned into a plurality of pressure-receiving regions. It results in a partitioning pattern of the hand. Optionally, the hand's surface may be identified with more than two categories of pressure regions, typically leading to a greater number of pressure-receiving regions in the partitioning such that unique needs (e.g., pressure required, comfort) of different parts of the hand may be better addressed.

After the hand's surface is partitioned, in a fourth step 204 of the method, an upper bound and a lower bound of pressure to be received from the pressure glove for each of the plurality of pressure-receiving regions are determined. In particular, determining such upper and lower bounds is according to at least (1) wound-related details of the hand including locations, conditions and maturation of potential hypertrophic scars formed or potentially formed on the hand and (2) comfort to portions of the hand without such potential hypertrophic scars. Thereby, the pressure glove is configured to discourage hypertrophic scar development while providing comfort to the patient. As mentioned above, the lower bound and the upper bound for a pressure-receiving region located on a wounded area of the hand may be set as 16.5 mmHg and 24.0 mmHg, respectively, while the lower bound for the one located on an intact area may be set to 0 mmHg. In general, the upper bound for a pressure-receiving region on the intact area may be set as long as comfort can be ensured. However, pressure induced at a pressure-receiving region on the wounded area is, to some extent, positively related to overall fabric tension controlled by, e.g., the reduction factor mentioned above, so that an actual pressure induced on the intact area can only be relatively lower than an actual pressure induced on the wounded area. It follows that a very low setting of the upper bound may not be practical for some pressure-receiving regions on the intact area.

In a fifth step 205 of the method, a partitioning pattern for partitioning the pressure glove into a plurality of custom pressure-applying fabric portions is determined. In particular, this partitioning pattern is obtained such that the partitioning pattern of the pressure glove is substantially similar to the partitioning patterning of the hand. Hence, the pressure glove is designed to comprise the plurality of custom pressure-applying fabric portions each of which directs a pressure onto one of the pressure-receiving regions of the hand with said directed pressure within the upper bound and the lower bound of pressure determined for said one of the pressure-receiving regions.

A sixth step 206 of the method is to produce a pressure glove having the custom pressure-applying fabric portions such that each of these fabric portions is configured to direct a pressure onto one of the pressure-receiving regions and within the upper bound and the lower bound of pressure determined therefor.

In producing the pressure glove, preferably, each of the pressure-applying fabric portions is fabricated by a fabric, and the pressure glove is fabricated with a size less than the hand's size by a reduction factor. A set of selected fabrics for fabricating the custom pressure-applying fabric portions is thereby obtained. Preferably, the sixth step 206 of the method comprises: a first sub-step 206a of determining the reduction factor and the set of selected fabrics; and a second sub-step 206b of fabricating the pressure glove according to (1) geometric details of the custom pressure-applying fabric portions, and (2) the set of selected fabrics. The reduction factor and the set of selected fabrics are determined by obtaining a computed pressure distribution of the hand's surface under a simulated condition of wearing the pressure glove, and by checking the computed pressure distribution against the upper bounds and the lower bounds for all the pressure-receiving regions, so as to ensure that each of the pressure-receiving regions receives a pressure within the upper bound and the lower bound both determined for said each of the pressure-receiving regions. The computed pressure distribution is obtained by analyzing the bio-mechanical model of the hand and a mechanical model of the pressure glove. The mechanical model of the pressure glove incorporates the geometric details of the custom pressure-applying fabric portions, and stiffness parameters of the fabrics selected for fabricating the custom pressure-applying fabric portions. As is mentioned above, the geometric details of the custom pressure-applying fabric portions provide at least a numerical description of shape and size of each of the custom pressure-applying fabric portions, and preferably this numerical description is specified for a two-dimensional plane.

It is an option that at least one of the custom pressure-applying fabric portions is fabricated by a multi-layer fabric comprising at least a first fabric layer and a second fabric layer. The first fabric layer and the second fabric layer overlie each other and are joined together. In addition, the first fabric layer and the second fabric layer are substantially different in free size before being joined together. As such, the sixth step 206 of the method further comprises an optional third sub-step 206c performed before the second sub-step 206b. In the third sub-step 206c, the free size of the first fabric layer and the free size of the second fabric layer are co-determined with the reduction factor and the set of selected fabrics according to the computed pressure distribution.

The present invention may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

What is claimed is:

1. A method for producing a therapeutic post-injury pressure glove configured to discourage hypertrophic scar development on a patient's hand having an injury, the pressure glove comprising a plurality of custom pressure-applying fabric portions that partition the pressure glove, the method comprising:
developing a bio-mechanical model of the patient's hand according to measured anatomical details of the hand including:
(a) 3D geometric modeling details of the hand; and
(b) stiffness parameters of one or more of the hand's anatomical structures over the hand's surface;
predicting one or more pressure distributions of the hand's surface under a simulated condition of wearing a reference glove made of a reference material by analyzing the bio-mechanical model of the hand and a mechanical model of the reference glove, each of the one or more predicted pressure distributions being obtained for a selected hand posture, wherein the mechanical model of the reference glove incorporates geometric details of the reference glove and stiffness parameters of the reference material;
identifying, by examining the one or more predicted pressure distributions, at least a plurality of high-pressure regions and a plurality of low-pressure regions on the hand's surface, so as to partition the hand's surface into a plurality of pressure-receiving regions, thereby resulting in a partitioning pattern of the hand;
determining an upper bound and a lower bound of pressure to be received from the pressure glove for each of the plurality of pressure-receiving regions according to at least:
(a) wound-related details of the hand including locations, conditions and maturation of potential hypertrophic scars formed or potentially formed on the hand; and
(b) comfort to portions of the hand without such potential hypertrophic scars;
thereby configuring the pressure glove to discourage hypertrophic scar development while providing comfort to the patient;
determining a partitioning pattern for partitioning the pressure glove into the plurality of custom pressure-applying fabric portions wherein the partitioning pattern of the pressure glove is substantially similar to the partitioning pattern of the hand, so that the pressure glove is designed to comprise the plurality of custom pressure-applying fabric portions each of which directs a pressure onto one of the pressure-receiving regions of the hand with said directed pressure within the upper bound and the lower bound of pressure determined for said one of the pressure-receiving regions; and
producing a pressure glove having the custom pressure-applying fabric portions such that each of the custom pressure-applying fabric portions is configured to direct a pressure onto one of the pressure-receiving regions and within the upper bound and the lower bound of pressure determined therefor.

2. The method of claim 1, wherein the reference glove is based on a glove pattern selected for making the pressure glove.

3. The method of claim 1, wherein the stiffness parameters of one or more of the hand's anatomical structures over the hand's surface comprise at least a tensile or compressive stiffness, a shear modulus and Poisson's ratio.

4. The method of claim 1, wherein the 3D geometric modeling details of the hand are acquired by a photogrammetric scanning system or other imaging system.

5. A therapeutic post-injury pressure glove formed by the method of claim 1.

6. The method of claim 1, each of the custom pressure-applying fabric portions being fabricated by a fabric, thereby resulting in a set of selected fabrics for fabricating the custom pressure-applying fabric portions, the pressure glove being fabricated with a size less than the hand's size by a reduction factor, wherein the step of producing a pressure glove having the custom pressure-applying fabric portions comprises:
determining the reduction factor and the set of selected fabrics by obtaining a computed pressure distribution of the hand's surface under a simulated condition of wearing the pressure glove, and by checking the computed pressure distribution against the upper bounds and the lower bounds for all the pressure-receiving regions, so as to ensure that each of the pressure-receiving regions receives a pressure within the upper bound and the lower bound both determined for said each of the pressure-receiving regions, wherein the computed pressure distribution is obtained by analyzing the bio-mechanical model of the hand and a mechanical model of the pressure glove, the mechanical model of the pressure glove incorporating geometric details of the custom pressure-applying fabric portions, and stiffness parameters of the fabrics selected for fabricating the custom pressure-applying fabric portions; and
fabricating the pressure glove according to the geometric details of the custom pressure-applying fabric portions, and the set of selected fabrics for fabricating the custom pressure-applying fabric portions.

7. A therapeutic post-injury pressure glove formed by the method of claim 6.

8. The method of claim 6, at least one of the custom pressure-applying fabric portions being fabricated by a fabric that is a multi-layer fabric comprising at least a first fabric layer and a second fabric layer, the first fabric layer and the second fabric layer overlying each other and being joined together, the first fabric layer and the second fabric layer being substantially different in free size before being joined together, wherein the step of producing a pressure glove having the custom pressure-applying fabric portions further comprises co-determining the free size of the first fabric layer and the free size of the second fabric layer with the reduction factor and the set of selected fabrics according to the computed pressure distribution.

9. A therapeutic post-injury pressure glove formed by the method of claim 8.

10. A therapeutic post-injury pressure glove personalized to a patient and configured to apply pressure onto a plurality of pressure-regions of the patient's hand having an injury such that each of the pressure-receiving regions receives a pressure within an upper bound and a lower bound both determined for said each of the pressure-receiving regions, wherein the pressure glove is characterized in that:
the pressure glove comprises a plurality of custom pressure-applying fabric portions each of which is configured to direct a pressure onto one of the pressure-receiving regions and is fabricated by a fabric, thereby resulting in a set of selected fabrics for fabricating the custom pressure-applying fabric portions;
the pressure glove is fabricated with a size less than the hand's size by a reduction factor; and
the reduction factor and the set of selected fabrics are determined by obtaining a computed pressure distribution of the hand's surface under a simulated condition of wearing the pressure glove, and by checking the computed pressure distribution against the upper bounds and the lower bounds for all the pressure-receiving regions, so as to ensure that each of the pressure-receiving regions receives a pressure within the upper bound and the lower bound both determined for said each of the pressure-receiving regions, wherein the computed pressure distribution is obtained by analyzing a bio-mechanical model of the hand and a mechanical model of the pressure glove, in which:
(a) the bio-mechanical model of the hand incorporates 3D geometric modeling details of the hand, and stiffness parameters of one or more of the hand's anatomical structures over the hand's surface; and
(b) the mechanical model of the pressure glove incorporates geometric details of the custom pressure-applying fabric portions, and stiffness parameters of the fabrics selected for fabricating the custom pressure-applying fabric portions;
and
the pressure glove is personalized to the patient by at least obtaining the 3D geometric modeling details of the hand and the stiffness parameters of one or more of the hand's anatomical structures over the hand's surface by physical measurement on the hand.

11. The pressure glove of claim 10, wherein at least two fabrics in the set of selected fabrics are substantially different in one or more aspects selected from composition, knitting structure, stress-strain and mechanical properties, comfort, air permeability, and deterioration of fabric elasticity, thereby allowing the pressure glove to be configured to achieve comfort and functional performance for the patient while discouraging hypertrophic scar development on the hand.

12. The pressure glove of claim 10, wherein at least one of the custom pressure-applying fabric portions is fabricated by a fabric that is a multi-layer fabric comprising at least a first fabric layer and a second fabric layer, the first fabric layer and the second fabric layer overlying each other and being joined together.

13. The pressure glove of claim 12, wherein the first fabric layer and the second fabric layer are substantially different in one or more aspects selected from composition, knitting structure, stress-strain and mechanical properties, comfort, air permeability, and deterioration of fabric elasticity.

14. The pressure glove of claim 12, wherein the first fabric layer and the second fabric layer are substantially similar in size.

15. The pressure glove of claim 12, wherein the first fabric layer and the second fabric layer are substantially different in free size before being joined together, thereby allowing a custom pressure-applying fabric portion fabricated by a multi-layer fabric having such first and second fabric layers to direct a different pressure onto a pressure-receiving region when compared to a custom pressure-applying fabric portion fabricated by a multi-layer fabric having a first fabric layer and a second fabric layer of equal free size.

16. The pressure glove of claim 15, wherein the free size of the first fabric layer and the free size of the second fabric layer are co-determined with the reduction factor and the set of selected fabrics according to the computed pressure distribution.

17. The pressure glove of claim 10, wherein the stiffness parameters of one ore more of the hand's anatomical structures over the hand's surface comprise at least a tensile or compressive stiffness, a shear modulus and Poisson's ratio.

18. The pressure glove of claim 10, wherein the 3D geometric modeling details of the hand are acquired by a photogrammetric scanning system or other imaging system for performing physical measurement on the hand.
